(19) United States
(12) Patent Application Publication
(10) Pub. No.: US 2016/0142478 A1
(43) Pub. Date: May 19, 2016

(54) COMMUNICATION MANAGEMENT SYSTEM

(71) Applicants: KABUSHIKI KAISHA TOSHIBA, Tokyo (JP); TOSHIBA SOLUTIONS CORPORATION, Kanagawa (JP)

(72) Inventors: Shingo MIYAZAKI, Fuchu, Tokyo (JP); Toshio OKAMOTO, Bunkyo, Tokyo (JP)

(21) Appl. No.: 14/900,853

(22) PCT Filed: Jun. 23, 2014
(86) PCT No.: PCT/JP2014/003337

§ 371 Date: Dec. 22, 2015

(30) Foreign Application Priority Data
Jun. 24, 2013 (JP) 2013-131786

(51) Int. Cl. H04L 29/08; G06F 19/00; A61B 5/00

(52) U.S. Cl. CPC H04L 67/1057; A61B 5/0022; A61B 5/4857; A61B 5/6889; H04L 67/12; G06F 19/3418; A61B 2503/08; A61B 2505/07

(57) ABSTRACT

[PROBLEM] To provide a communication management system dynamically forming a communication network between users based on user's health condition and living behaviors to support more active communication between the users relating to the health or life.

[SOLVING MEANS] A computer system according to an embodiment is a communication management system configured to provide communication network information for users, and includes a storage apparatus configured to store for each of the users physiological information and/or living behavior information of the user, acquired dynamically in daily life, and a control apparatus configured to produce the communication network information being associated with other users to a first user based on the physiological information and/or living behavior information of the first user. The control apparatus is configured to produce the communication network information by using each of the dynamically acquired physiological information and/or living behavior information of the first user as a network formation element.

### TECHNICAL FIELD

The present invention relates to a technology for dynamically forming a communication network between users from user's health condition and living behaviors to support more active communication between the users relating to the health or life.

### BACKGROUND ART

Technologies have conventionally been proposed for allowing the health condition of an elderly person to be watched by a family member or medical agency. Technologies have also been proposed for connecting a terminal apparatus provided for an elderly person to a terminal apparatus provided for a family member or a medical agency over a network to have communication between the elderly person and the family member through the terminal apparatuses.

### PRIOR ART DOCUMENT

### Patent Document

[Patent Document 1] Japanese Patent Laid-Open No. 2010-072720

### DISCLOSURE OF THE INVENTION

### Problems to be Solved by the Invention

It is an object of the present invention to provide a communication management system capable of dynamically forming a communication network between users from the user's health condition and living behaviors to support more active communication between the users relating to the health or life.

### Means for Solving the Problems

A computer system according to an embodiment is a communication management system connected to a plurality of information terminal apparatuses and configured to provide communication network information for communication of users between the plurality of information terminal apparatuses, and includes a storage apparatus configured to store for each of the users physiological information and/or living behavior information of the user, acquired dynamically in daily life, and a control apparatus configured to produce the communication network information being associated with other users to a first user based on the physiological information and/or living behavior information of the first user, wherein the control apparatus is configured to produce the communication network information by using each of the dynamically acquired physiological information and/or living behavior information of the first user as a network formation element.

### BRIEF DESCRIPTION OF THE DRAWINGS

FIG. 1 A diagram showing an exemplary system configuration of a communication management system according to Embodiment 1.

FIG. 2 A block diagram showing exemplary components of a management apparatus (communication management apparatus), a physiological information measurement apparatus, a living behavior acquirement apparatus, and an information terminal apparatus according to Embodiment 1.

FIG. 3 A diagram showing exemplary network definition information (network formation element) and exemplary group information (types) of data class for use in forming a communication network between users according to Embodiment 1.

FIG. 4 A diagram showing a flow of communication network production processing according to Embodiment 1.

FIG. 5 A diagram showing a detailed flow of the communication network production processing in the present embodiment.

FIG. 6 An example of disclosure/non-disclosure setting information of physiological information, living behavior situation and the like in the communication network according to Embodiment 1.

FIG. 7 A diagram showing an exemplary blood pressure communication network according to Embodiment 1.

FIG. 8 A diagram showing an exemplary communication network for each blood pressure type (for each group of data class) according to Embodiment 2.

FIG. 9 A diagram showing an exemplary blood pressure communication network according to Embodiment 1 and showing an exemplary network including blood pressure status changes between preceding and present measurements.

FIG. 10 A diagram showing a flow of processing of producing the communication network including the status changes according to Embodiment 1.

FIG. 11 A diagram showing an exemplary communication network of daily blood pressure change display type according to Embodiment 1.

FIG. 12 A diagram showing an exemplary living behavior communication network according to Embodiment 1.

FIG. 13 A diagram of an exemplary living sound communication network according to Embodiment 1.

FIG. 14 A diagram of an exemplary life rhythm communication network according to Embodiment 1.

FIG. 15 A diagram showing a flow of life rhythm communication network production processing according to Embodiment 1.

FIG. 16 A diagram of life rhythm pattern definition information according to Embodiment 1.

FIG. 17 A diagram showing life rhythm changes and pattern changes according to Embodiment 1.

FIG. 18 A diagram showing exemplary changes in health and communication according to Embodiment 1.

FIG. 19 A diagram showing a flow of communication network production processing (based on ranking) according to Modification 1.

FIG. 20 A diagram showing a flow of communication network production processing (based on schedule) according to Modification 2.

FIG. 21 A diagram showing exemplary display of a plurality of communication network groups according to Embodiment 1.

FIG. 22 A diagram showing exemplary display of members of a selected communication network group according to Embodiment 1.

### MODE FOR CARRYING OUT THE INVENTION

A preferred embodiment will hereinafter be described with reference to the drawings.

### Embodiment 1

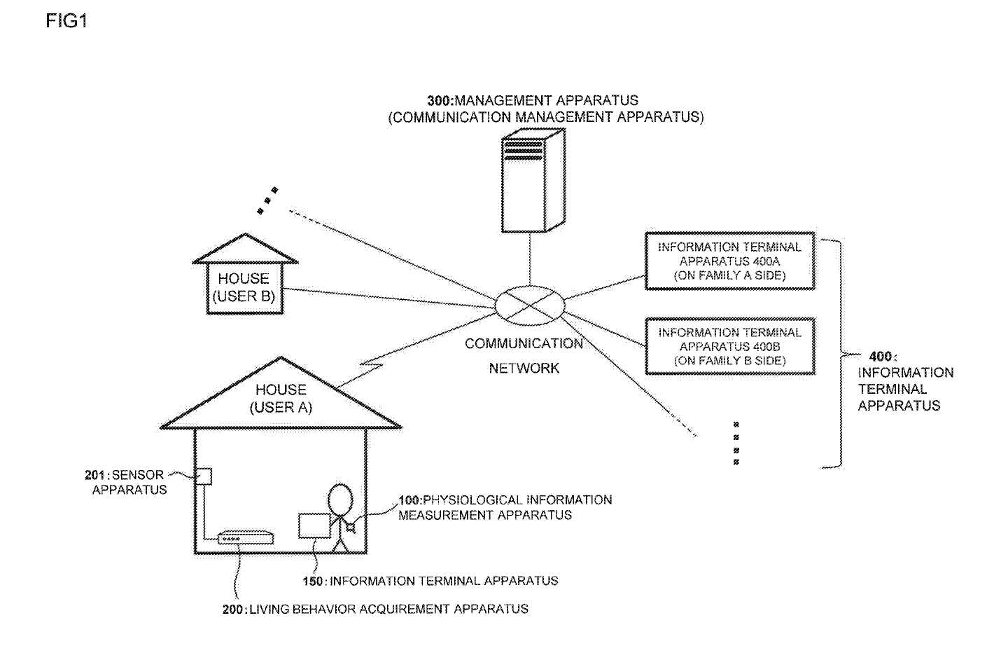

FIGS. 1 to 22 are diagrams showing an exemplary system configuration of a communication management system according to Embodiment 1. The communication management system of the present embodiment is a computer system for finding the daily health condition and living behaviors of a user and dynamically forming a communication network from the found health condition or living behaviors to support more active communication between the users relating to the health or life.

The communication management system of the present embodiment finds the daily health condition and living behaviors of the user and thus can be configured, for example, to include a computer system for allowing a user such as an elderly person and a live-alone to be watched by a user's family member, relative, or a public agency (such as a medical agency and a care agency).

Although the present embodiment is described in an aspect of the communication management system including a watching system by way of example, the communication management system may be implemented as a computer system independent of the watching system.

As shown in FIG. 1, when the watching system is applied, the system can be configured to connect a user to be watched such as an elderly person with a user's family member in a one-to-one relationship or to connect an elderly person with a plurality of family members or a plurality of public agencies in a one-to-many relationship. Alternatively, a user such as an elderly person can be connected only to the communication management system without connection to any family member or public agency, for example.

As shown in FIG. 1, the communication management system includes various apparatuses provided on a user side such as an elderly person (person to be watched), a management apparatus (communication management apparatus) 300, and an information terminal apparatus 400 provided on a watcher side such as a family member who watches the user, all of which are connected to each other over a communication network.

Various apparatuses provided on the user side include a physiological information measurement apparatus 100 for acquiring physiological information of the user, a living behavior acquirement apparatus 200 provided with a sensor apparatus 201 usually installed in a house of the user for primarily acquiring information about living behaviors of the user in the house, and an information terminal apparatus 150 operable by the user.

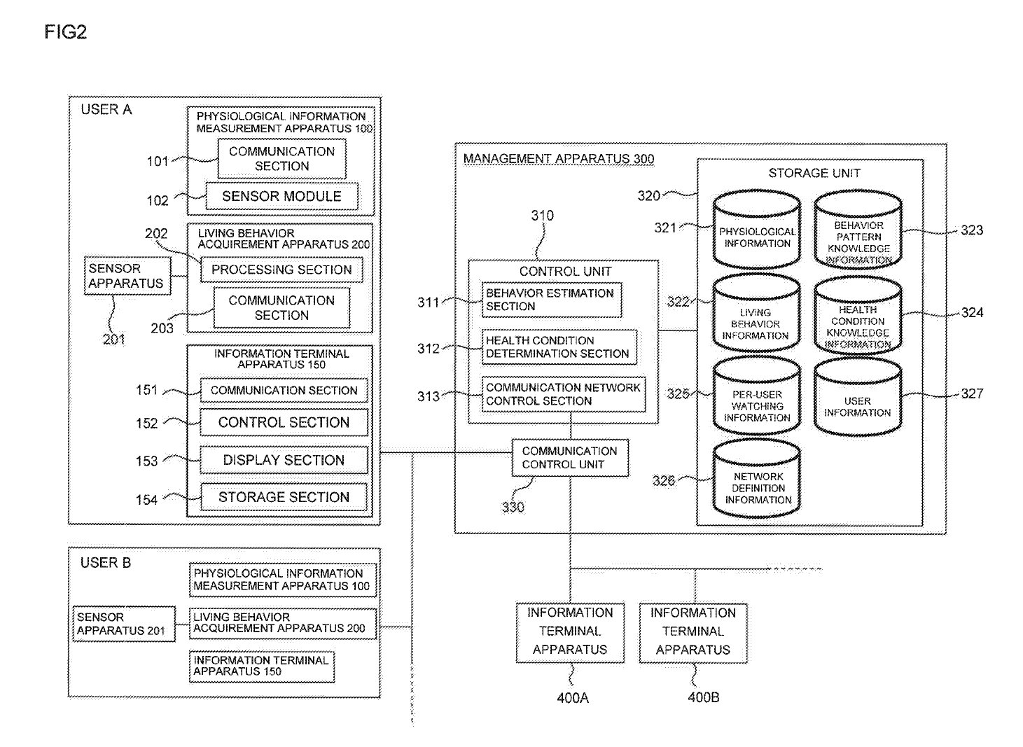

FIG. 2 is a block diagram showing components of the apparatuses constituting the communication management system.

As shown in FIG. 2, the physiological information measurement apparatus 100 is a wearable biometric sensor put on the user's body to measure the physiological information. Examples of the physiological information measurement apparatus include a head-mount type and a wrist type put on a user's wrist, and a known physiological information measurement apparatus can be used. The physiological information measurement apparatus 100 includes a communication section 101 for controlling communication such as wired communication, wireless communication, and infrared communication and can communicate data with the management apparatus 300 and another communication device connected to the management apparatus 300 (a cellular phone, a multi-function cellular phone, a personal digital assistant (PDA), a tablet terminal apparatus and the like owned or carried by the user).

Even when the user wearing the physiological information measurement apparatus 100 is outside the house, the communication section 101 for controlling communication such as wireless communication and infrared communication enables data communication with the management apparatus 300 and another communication device installed in the house connected to the management apparatus 300. When the user carries a cellular phone having a GPS function, the user can be located to acquire information indicating where the user goes as well as living behavior information while the user is at home or out.

The physiological information measurement apparatus 100 includes a plurality of sensor modules 102. The sensor modules are sensor apparatuses for measuring various types of physiological information including the heart rate, pulse, blood pressure (pulse waves), and body temperature. Each sensor module can be attached to part of the body such as a fingertip, chest, and head to measure the physiological information including the pulse, heart rate, Galvanic Skin Response (GSR), body temperature (body surface temperature), blood pressure, electrocardiogram, and brain waves. The measured physiological information can include a biological sound such as swallowing sound and heartbeat sound, and user's facial expression. In this case, the physiological information measurement apparatus 100 can have an imaging function provided by a camera or the like. The physiological information measurement apparatus 100 can also have a living behavior information acquiring function, later described, provided by an acceleration sensor, pedometer, sound-collecting microphone or the like.

The physiological information measurement apparatus 100 does not necessarily include the sensor modules integrated into one unit for measuring all types of physiological information, and a plurality of apparatuses may be provided separately for the respective measurement functions (sensor modules). Thus, a plurality of physiological information measurement apparatuses can measure a plurality of types of physiological information, or a single physiological information measurement apparatus can measure a plurality of types of physiological information. Of the plurality of types of physiological information, the pulse and the body temperature may be measured by a single sensor module, for example. A single sensor module may measure a single or a plurality of types of physiological information.

The physiological information measurement apparatus 100 can include not only the wearable biometric sensor wearable on the user's body but also a stationary physiological information measurement apparatus fixed in the user's house. Examples thereof include a manometer and a weight scale. In this case, the manometer or the weight scale can be provided with a communication function to allow data communication with the management apparatus 300 via another communication device installed in the house connected to the management apparatus 300 or the wearable physiological information measurement apparatus 100.

The stationary physiological information measurement apparatus 100 may be used by a plurality of users living in the same house. Thus, physiological information of a user can be transmitted to the management apparatus 300 together with authentication information such as the user's ID to manage measured physiological information for each user. The stationary physiological information measurement apparatus such as the manometer and the weight scale may transmit the physiological information together with authentication information such as the user's ID directly to the management apparatus 300 by using the communication function. The communication may also be performed over a medium such as a cellular phone capable of communication with the management apparatus 300 or the Internet (for example, with Wi-Fi®), although such a medium is not installed in the house connected to the management apparatus 300.

The physiological information measured by the physiological information measurement apparatus 100 is associated with a measurement time and is sequentially transmitted to the management apparatus 300. For example, the physiological information measurement apparatus 100 or another communication device (such as the information terminal apparatus 150, later described) may temporarily store a plurality of physiological information pieces acquired in temporal sequence and the management apparatus 300 may collectively acquire the plurality of physiological information pieces at predetermined points in time. In this case, the physiological information does not need to be always transmitted, so that the amount of data communication can be limited.

The living behavior acquirement apparatus 200 is connected to the sensor apparatus 201 installed in the user's house such as a human detection sensor, infrared sensor, pressure sensor, temperature sensor, camera (imaging apparatus), sound-collecting microphone, consumed power measurement sensor, tap water amount measurement sensor, and gas measurement sensor, and acquires, as living behavior information, various types of information detected by the sensor apparatus 201 that correspond to the behaviors of the user.

In the living behavior acquirement apparatus 200, a processing section 202 processes the detection information output from the sensor apparatus 201, outputs the living behavior information of the user in temporal sequence, and transmits the information to the management apparatus 300 via a communication section 203. The living behavior information can be acquired sequentially in response to sensing by the sensor apparatus 201 or at a predetermined point in time in association with another sensor. For example, video or audio can be started to be collected at the time when the human detection sensor detects the user.

The living behavior acquirement apparatus 200 can be connected to an electrical appliance such as an air conditioner, heater, washing machine, cleaner, dryer, and television apparatus in the user's house, an electrical/gas appliance such as a wattmeter, water heater, IH cooking heater, and gas cooking stove, or a meter for water and sewage through wired or wireless communication, and can acquire the usage of a product or equipment or on/off operation of a switch, by way of example, as the living behavior information of the user.

As described above, part of the function of acquiring the living behavior information representing the life of the user may be provided for the physiological information measurement apparatus 100. In other words, the physiological information measurement apparatus 100 can be configured as the living behavior acquirement apparatus 200, and vice versa (the living behavior acquirement apparatus 200 can be responsible for the function of the physiological information measurement apparatus). Depending on how to acquire the physiological information and the living behavior information, the physiological information and the living behavior information can be acquired by a single apparatus or by each of independent apparatuses.

The living behavior acquirement apparatus 200 may be formed integrally with the sensor apparatus 201. Specifically, each sensor apparatus 201 can have a communication function to perform data communication with the management apparatus 300, can have a function of computing and processing detected living behavior information into predetermined data or data format, or can have a function of adding the measurement time.

Another example of the living behavior acquirement apparatus 200 is the information terminal apparatus 150 on the user side. Information acquired through input operation by the user on the information terminal apparatus 150 (such as selection of a “took medicine” button on a medicine-taking check screen) may be collected by the management apparatus 300 as the living behavior information. The living behavior information can also be acquired by a camera (imaging apparatus) or a sound-collecting microphone provided for the information terminal apparatus 150.

The information terminal apparatus 150 is, for example, a tablet terminal apparatus (tablet computer) used by the user. The information terminal apparatus 150 is owned by each of a plurality of users who use services offered by the management apparatus 300, and can be used in or out of the house. The user may have a plurality of information terminal apparatuses 150.

The information terminal, apparatus 150 according to the present embodiment includes a communication section 151 for controlling communication with the management apparatus 300 over the communication network, a control section 152 for performing display control and input control through a display section 153, the display section 153 including a touch panel serving as an input apparatus, and a storage section 154. The information terminal apparatus 150 can also include a speaker, a sound-collecting microphone, various operation buttons and the like.

The information terminal apparatus 150 can be connected to information terminal apparatuses 150 of other users connected to the management apparatus 300 over the communication network (including an IP network and a telephone network). In this case, the information terminal apparatus 150 can be connected to the other information terminal apparatuses 150 via the management apparatus 300 or can be connected directly to the other information terminal apparatuses 150. The control of these connections is performed by the communication section 151 to provide the wireless or wired communication function.

The information terminal apparatus 150 can perform wired or wireless communication with the physiological information measurement apparatus 100 and the living behavior acquiring apparatus 200 (including the sensor apparatus 201). As described above, the information terminal apparatus 150 can perform communication with the physiological information measurement apparatus 100 and the living behavior acquiring apparatus 200 to receive the physiological information and the living behavior information. The Information terminal 150 serving as a communication apparatus for transmitting the physiological information and the living behavior information to the management apparatus 300 can add header information such as the user ID and the point in time to the physiological information and the living behavior information before transmission to the management apparatus 300 and can function as a storing section for storing the physiological information and the living behavior information in the storage section 154 in cooperation with or independently of the management apparatus 300.

The information terminal apparatus 150 can display a predetermined menu screen on the display section 153 and provide functions associated with input operation by the user on the menu screen. Examples of those functions include an e-mail function (including a handwriting input function), telephone call function (including a videophone), a function of connection to an Internet site, a function of displaying the physiological information and health information of the user, and a function of inputting confirmation of medicine taking. To provide these functions, various arbitrary screens are displayed on the display section 153 to allow input control in response to input operation through the touch panel or display control of various screens. Screen information can be stored previously in the storage section 154, or screen information (or screen information including display information) can be acquired from the management apparatus 300 each time it is required.

The information terminal apparatus 150 of the present embodiment displays a predetermined communication network screen on the display section 153 based on communication network information provided by the management apparatus 300 to provide a function of communication with other users displayed on the communication network screen. For example, icons of other users can be displayed on the communication network screen, and when the user selects one of them, the function of transmitting an e-mail or message or making a call can be activated.

As described above, the information terminal apparatus 150 of the present embodiment can be used as an input/output interface for performing communication with the management apparatus 300 to watch the health condition and living behaviors and can serve as a communication apparatus for exchange of messages or conversations with other users of system members or users other than the system members.

Next, the management apparatus 300 is described. The management apparatus 300 can store the physiological information and living behavior information of the user acquired from the physiological information measurement apparatus 100 and the living behavior acquirement apparatus 200, respectively, estimate a living behavior situation of the user from the living behavior information of the user, and determine (estimate) the user's health condition based on the physiological information.

The management apparatus 300 includes a control unit 310, a storage unit 320, and a communication control unit 330. The communication control unit. 330 controls data communication with the physiological information measurement apparatus 100, the living behavior acquirement apparatus 200, and the information terminal apparatus 150 of the user, and controls communication with the information terminal apparatus 400 on the watching side.

The control unit 310 includes a behavior estimation section 311 for estimating the living behavior situation of the user based on the living behavior information of the user, a health condition determination section 312 for determining the user's health condition based on the physiological information of the user, and a communication network control section 313.

The storage unit 320 stores user's physiological information 321 received from the physiological information measurement apparatus 100 for each user. For example, the storage unit 320 can store each physiological information with the associated time and measured value in temporal sequence. The storage unit 320 also stores user's living behavior information 322 received from the living behavior acquirement apparatus 200 for each user. For example, the storage unit 320 stores detection information from each sensor apparatus 201 together with the detection time in temporal sequence.

Behavior pattern knowledge information 323 is information including previously specified behavior patterns corresponding to detection information detected by the sensor apparatus 201. The behavior estimation section 311 refers to the behavior pattern knowledge information 323 and matches a behavior pattern with corresponding living behavior information 322 to identify the living behavior situation of the user.

For example, when detection information from a pressure sensor installed at a place for sleeping such as a mattress or a bed changes from a pressure corresponding to the user's weight to a lower pressure, rising of the user can be known. In this case, an acceleration sensor may determine the user's posture such that the rising of the user can be known based on a plurality of living behavior information pieces from the pressure sensor and the acceleration sensor. In the behavior pattern knowledge information 323, for example, the user's behavior of “rising” is previously associated with living behavior information (such as a pressure change detected by the pressure sensor and a posture detected by the acceleration sensor). The behavior estimation section 311 refers to the behavior pattern knowledge information 323, matches it with living behavior information acquired in temporal sequence, and identifies the living behavior situation of the user. The behavior estimation section 311 can arrange and store the estimated living behavior situations in temporal sequence for each user in the storage unit 320.

Sound information acquired as the living behavior information can be used to find a living behavior such as “sneezing,” “coughing,” “passing gas,” “belching,” “laughing,” and “crying” based on the biological sound of the user. For example, sound information (user's voice or sound or sample sound) corresponding to each living behavior can be previously stored and matched with the acquired sound information to estimate the living behavior situation of the user.

The sound information can be combined with another living behavior information to find a user's behavior such as falling or hitting a door or wall. For example, the user's posture is acquired by the acceleration sensor at the same time as the acquisition of sound information, and from a previously sampled hitting sound and the information about user's posture (such as crouching), the user's behavior of “falling” can be known.

User's behaviors such as “cleaning,” “washing,” “washing dishes,” “using dryer,” and “watching television” can also be known. For example, the behavior estimation section 311 can acquire, as living behavior information, a signal indicating that the washer or cleaner is operational acquired by the living behavior acquirement apparatus 200, or can acquire, as living behavior information, the continuous use situation of water from the water meter. The behavior estimation section 311 can also acquire on/off operation of the switch of the dryer or television as living behavior information.

The behavior estimation section 311 may acquire sound information together with the living behavior information such as the operational state or the on/off of the switch of the electric appliance acquired by the living behavior acquirement apparatus 200 and use them in combination. For example, when the switch of the cleaner is on and the driving sound of the cleaner is sensed from the sound information, it can be estimated that the user is “cleaning.” When the switch of the television is on and laughter or cry is acquired as the sound information, the user's behavior “watching television” can be known.

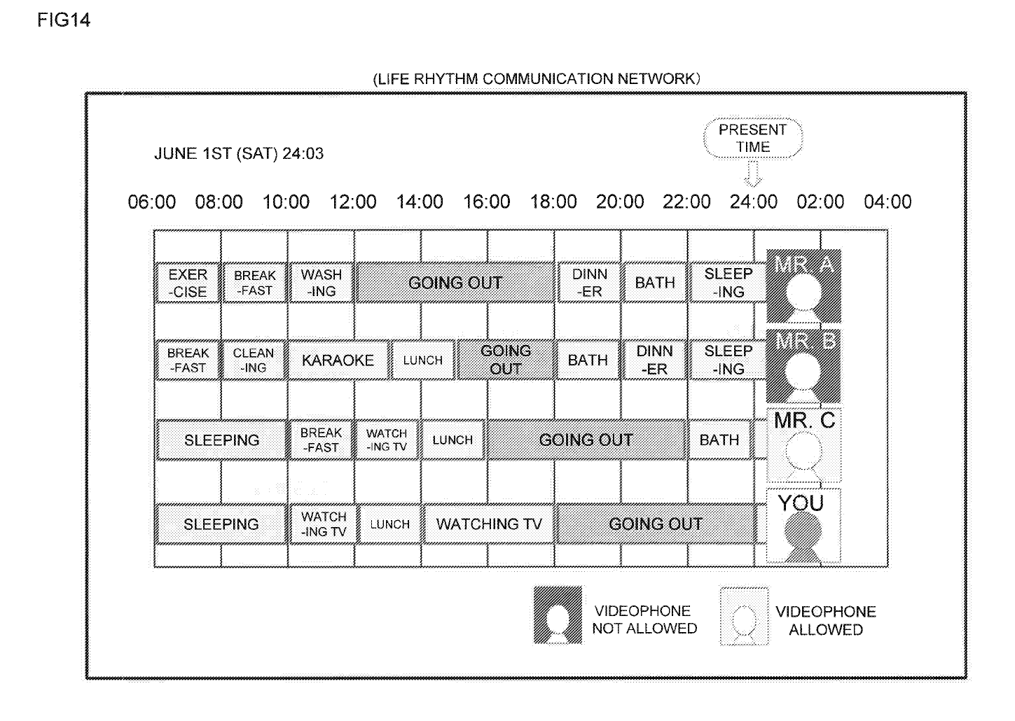

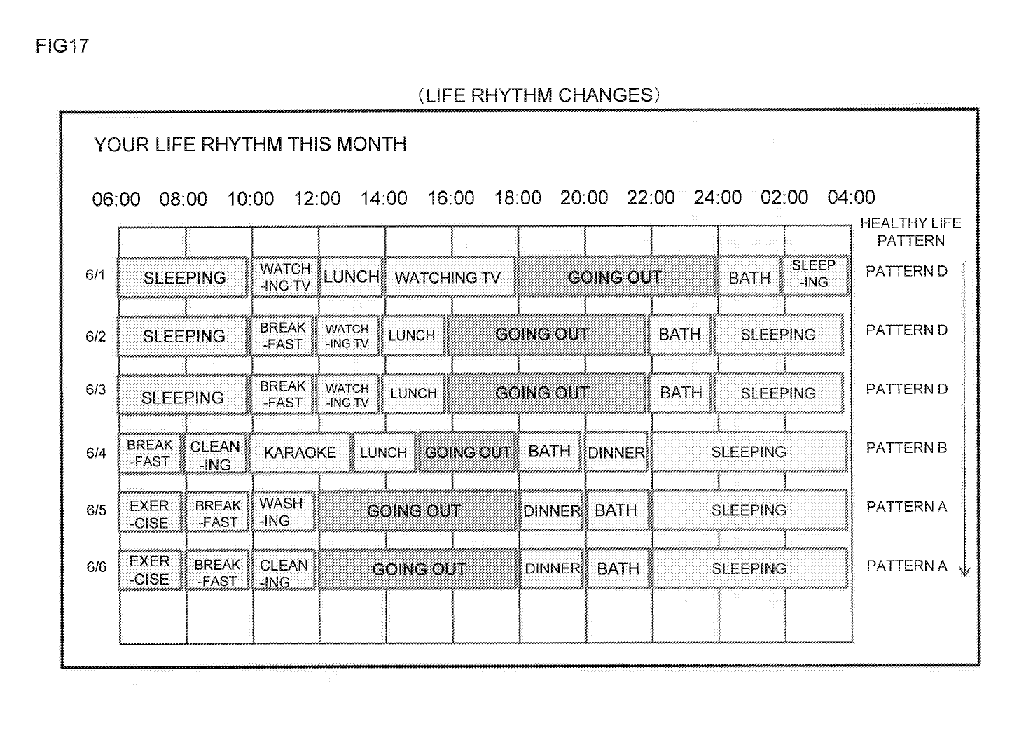

The obtained living behavior situations of the user can be arranged in temporal sequence to find the rhythm of the user's life (see FIG. 14). The behavior estimation section 311 can produce life rhythm information including a plurality of estimated living behavior situations for one day arranged in temporal sequence, and can also produce life rhythm change information including an array of daily life rhythms (see FIG. 17).

The living behavior situation and life rhythm of the user found from the living behavior information 322 acquired in the living behavior acquirement apparatus 200 are stored in temporal sequence as per-user watching information 325 in the storage unit 320. The behavior estimation section 311 uses the living behavior information 322 acquired in real time over the communication network and refers to the behavior pattern knowledge information 323 to find (produce) the living behavior situation of the user sequentially or at predetermined points in time, and stores the living behavior situation in temporal sequence as the watching information 325 in the storage unit 320. The behavior estimation section 311 also produces the life rhythm information included in the watching information 325 by arranging the living behavior situations stored in the watching information 325 in temporal sequence into a predetermined format sequentially or at predetermined points in time.

The behavior estimation section 311 and the life pattern knowledge information 323 included in the management apparatus 300 may be included in the information terminal apparatus 150 instead. When the information terminal apparatus 150 includes the behavior estimation section 311 and the life pattern knowledge information 323, the behavior estimation section 311 of the information terminal apparatus 150 refers to the life pattern knowledge information 323 and matches it with the living behavior information 322 acquired from the living behavior acquirement apparatus 200 through the communication section 151 to identify the living behavior situation of the user. The information terminal apparatus 150 stores the living behavior information 322 acquired from the living behavior acquirement apparatus 200 and the living behavior situation determined by the behavior estimation section 311 in the storage section 154 in association with the user information, or transmits the information from the communication section 151 to the management apparatus 300 and stores the information in the storage unit 320 of the management apparatus 300.

Next, the health condition determination section 312 is described. Health condition knowledge information 324 is determination information for determining the user's health condition from the physiological information 321.

For example, threshold values of blood pressure and body temperature are stored as health condition knowledge. The health condition determination section 312 refers to the health condition knowledge information 324 to determine whether the physiological information 321 is beyond determination values (upper limit and lower limit) in each physiological information or falls within the range from the upper limit to the lower limit specified as the determination values.

For example, when it is determined that none of physiological information pieces are beyond the determination values (all fall between the determination values determined to be normal), the health condition determination section 312 determines that the user's health condition is “normal.” When any physiological information piece is beyond the determination values determined to be normal but is not beyond determination values determined to be abnormal, that is, it is not determined to be normal or abnormal, the health condition determination section 312 determines that the user's health condition is “non-normal” as a health condition between “normal” and “abnormal.” When it is determined that any one physiological information piece is beyond the determination values (falls between the determination values determined to be abnormal), the health condition determination section 312 determines that the user's health condition is “abnormal.”

Each of the determined “normal,” “non-normal,” and “abnormal” health conditions can be subdivided. For example, the “normal” can be determined in subdivisions of “good” and “so-so.” Determination values for each of “good” and “so-so” can be stored as the health condition knowledge information. Similarly, the “abnormal” health condition can be subdivided into “mild” and “severe,” for example. Such arbitrary statuses can be associated with the determination values for determining the health condition, so that the health condition can be determined as an arbitrary status from the physiological information relating to the living behavior situation estimated from the behavior information 322.

The health condition determined by the health condition determination section 312 is stored in the watching information 325 for each user. The determined health condition can be stored in association with the living behavior situation of the user. For example, the living behavior situation of “rising” can be associated with the “normal” health condition determined from physiological information to allow the health condition to be known in association with the living behavior situation in temporal sequence.

Each time the living behavior situation is estimated, the health condition determination section 312 can determine the user's health condition from the physiological information associated with the estimated living behavior situation, and thus can associate determined health conditions with respective living behavior situations. A plurality of determined health conditions in the morning, afternoon, or a day can be averaged, and the averaged health condition may be determined once a day or once in the morning and afternoon.

In the processing of determining the user's health condition by the health condition determination section 312 in the present embodiment, the health condition can be known only from the physiological information 321. Alternatively, the living behavior situation of the user estimated from the living behavior information 322 can be taken into account such that the user's health condition is determined by using both the physiological information 321 and that living behavior situation.

For example, physiological information measured during climbing of stairs is physiological information of the user under an exercise load greater than at rest, so that the user's health condition may not be obtained (determined) accurately. To address this, when a movement of the user is detected in order by a human detection sensor installed at the bottom of the stairs and then a human detection sensor installed at the top of the stairs, the living behavior situation of the user can be determined to be “climbing of stairs.” Once the living behavior situation of climbing of stairs is obtained in determining the health condition, a finally determined health condition is “normal” or “non-normal” even when the “abnormal” health condition is formally determined from the determination information. In the determination of the user's health condition, either of the physiological information or the living behavior information may be used first.

Since the management apparatus 300 is connected to the information terminal apparatus 150 operable by the user over the communication network, the control unit 310 can provide the information terminal apparatus 150 with the obtained living behavior information (living behavior situation), life rhythm, physiological information, and health condition of the user to allow visual recognition (notification) or auditory recognition (sound output).

Since the management apparatus 300 is connected to the information terminal apparatus 400 of the watcher who watches the user over the network, the control unit 310 similarly can provide the information terminal apparatus 400 with the obtained living behavior information, life rhythm, physiological information, and health condition of the user to allow visual recognition by a family member or the like on the watcher side.

Rules of screen display and notification can be previously stored in association with the respective health conditions in the storage unit 320, and the management apparatus 300 can perform control such that a predetermined screen or notification associated with the user's health condition determined by the health condition determination section 312 is displayed in the information terminal apparatus 400 on the watcher side or in a medical agency.

The health condition determination section 312 and the health condition knowledge 324 included in the management apparatus 300 may be included in the information terminal apparatus 150 instead. When the information terminal apparatus 150 includes the health condition determination section 312 and the health condition knowledge 324, the health condition estimation section 312 of the information terminal apparatus 150 refers to the health condition knowledge 324 and determines the user's health condition from the physiological information 321 acquired from the physiological information measurement apparatus 100 through the communication section 154. The information terminal apparatus 150 stores the physiological information 321 acquired from the physiological information measurement apparatus 100 and the user's health condition determined by the health condition determination section 312 in the storage section 154 in association with the user information, or transmits the information from the communication section 151 to the management apparatus 300 and stores the information in the storage unit 320 of the management apparatus 300.

Next, description is made of the formation of the communication network in the present embodiment and the function of communication between a plurality of users with the formed communication network.

In the present embodiment, the communication network control section 313 is provided in order to dynamically form the communication network between users from the physiological information (including health data or health condition found from the physiological information) and living behavior situation of the user to support more active communication between users relating to health and life.

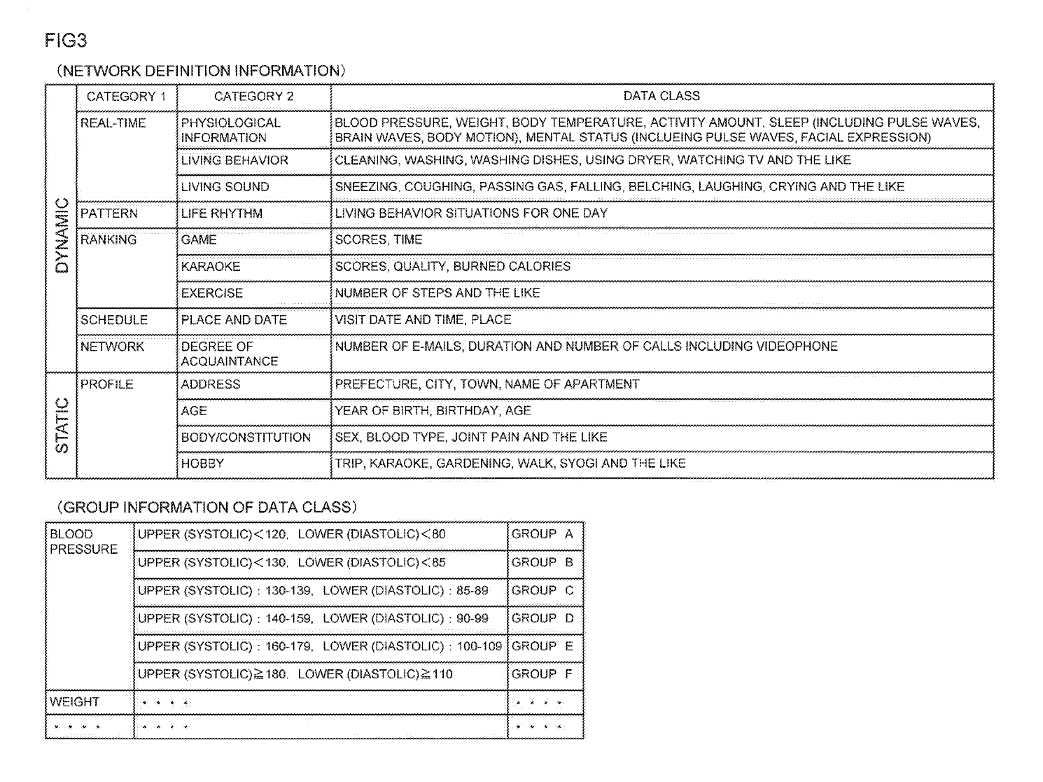

The communication network control section 313 uses network definition information shown in FIG. 3 as a communication network formation element to produce a communication network based on physiological information and/or living behavior information dynamically acquired in daily life for each group of users, and uses the dynamically acquired physiological information and/or living behavior information of the user as the network formation element to produce communication network information. The produced communication network information is provided as a place for communication between users through the information terminal apparatuses 150.

In FIG. 3, the network definition information 326 includes dynamic definition information and static definition information. The dynamic definition information includes “real time,” “pattern,” “ranking,” “schedule” and the like in a first category. The static definition information includes “profile” of each user, “degree of acquaintance” on the network and the like in the first category.

The “real time” in the first category of the dynamic definition information is linked to a plurality of items in a second category at a lower level and includes, in the second categories, the physiological information, living behavior (living behavior situation), and biological sound of the user acquired by the physiological information measurement apparatus 100 and the living behavior acquirement apparatus 200.

The second category includes a plurality of data classes. The “physiological information” is associated with data classes including blood pressure, weight, body temperature and the like. These data classes correspond to each information stored in the physiological information 321, living behavior information 322, and per-user watching information 325 in the present embodiment. In other words, each information in the physiological information 321, living behavior information 322, and per-user watching information 325 stored in the storage unit 320 is linked to each data class of the network definition information.

The “pattern” includes life rhythm information in the second category and represents the daily life rhythm of the user stored in the per-user watching information 325. The per-user watching information 325 includes daily life rhythm information of each user for a predetermined time period stored as the data class “life rhythm.”

The “ranking” and “schedule” in the first category are different from the physiological information, living behavior, and living sound of the user, and for example, correspond to information representing daily actions of the user based on hobbies and tastes such as records of games and karaoke played through the information terminal apparatus 150 or exercise records input to the information terminal apparatus 150. The data classes thereof include the scores of games and karaoke, the number of steps and the like. Since these information pieces change depending on daily actions of the user, they are classified as the dynamic network definition information.

The “network” in the first category includes the “degree of acquaintance” representing the strength of ties (degree of closeness) between users. For example, the degree of acquaintance can include the number of e-mails between users, and the duration and number of calls over the telephone or videophone in the data class. As the degree of communication is higher, the degree of acquaintance between the users is higher. Since the degree of acquaintance changes depending on the degree of communication, the degree of acquaintance is classified as the dynamic network definition information.

The “profile” in the first category of the static definition information corresponds to attribute information of the user and is previously stored as the user information 327 in the storage unit 320. The “profile” includes “address,” “age,” “body,” “constitution,” “hobby” and the like in the second category, and is personal information registered arbitrarily by the user. This information can be input and changed, for example through a predetermined registration screen on the information terminal apparatus 150.

The data class can be associated with group information. As shown in a lower part of FIG. 3, the data class “blood pressure”, for example, can be subdivided into a plurality of blood pressure types based on blood pressure values. Threshold values of each blood pressure group can be set arbitrarily.

The group information is definition information for grouping the physiological information 321 into a plurality of types and is used in the physiological information 321, living behavior information 322, and per-user watching information 325. Thus, the physiological information 321 and the like of each user can be previously stored in association with such a group (for example, blood pressure types) according to each group definition.

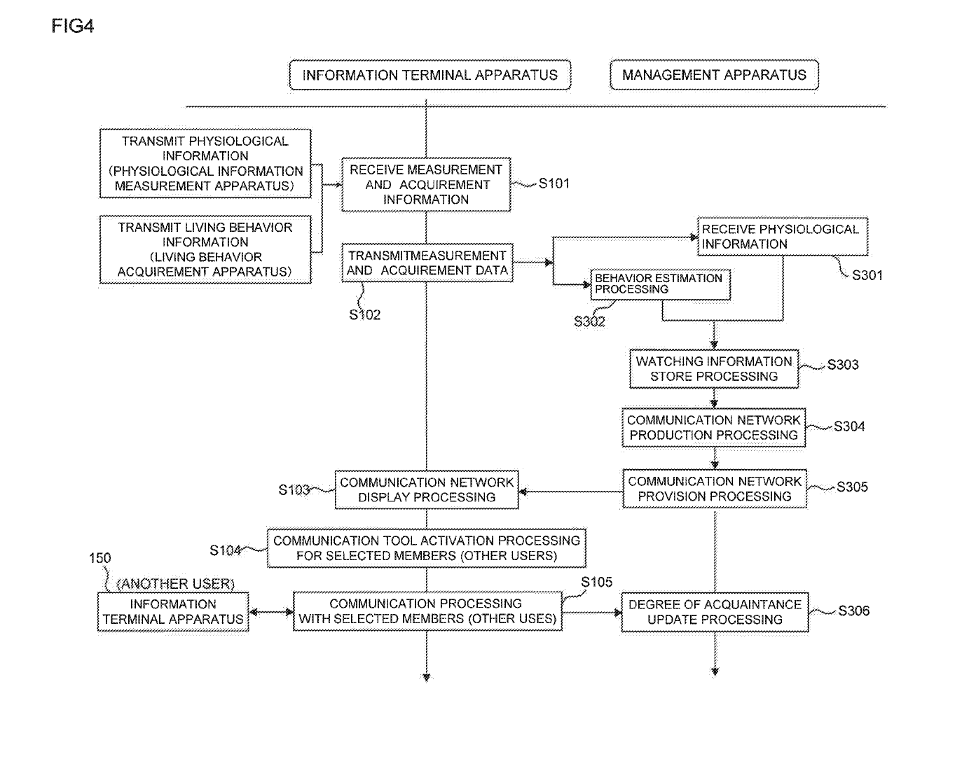

FIG. 4 is a diagram showing a flow of communication network production processing in the present embodiment. The example of FIG. 4 shows an aspect in which the information terminal apparatus 150 transmits the physiological information measured by the physiological information measurement apparatus 100 and the living behavior information acquired by the living behavior acquirement apparatus 200 to the management apparatus 300.

As shown in FIG. 4, when the information terminal apparatus 150 receives the physiological information from the physiological information measurement apparatus 100 and/or the living behavior information from the living behavior acquirement apparatus 200 (S101), the information terminal apparatus 150 transmits the physiological information and/or the living behavior information (hereinafter referred to collectively as physiological information or the like) to the management apparatus 300 (S102).

The management apparatus 300 receives the physiological information or the like (S301, S302) and stores the physiological information or the like in the storage unit 320 for each user. The management apparatus 300 stores the received physiological information in the storage unit 320 for each data class. The management apparatus 300 (control unit 310) refers to the group definition information (in the lower part of FIG. 3) associated with each data class to determine the group information (for example, blood pressure type) of the physiological information or the like. The physiological information or the like having the determined group information added thereto can be stored in the storage unit 320.

The management apparatus 300 can perform behavior estimation processing upon reception of the living behavior information to identify (determine) and store the living behavior situation of the user in the storage unit 320 as the per-unit watching information 325. The obtained living behavior situation may also be stored in the living behavior information 322.

As described above, the management apparatus 300 can also perform user's health condition determination processing upon reception of the physiological information or the like. In this case, the determined user's health condition is stored for each user as the per-user watching information 325, and the control unit 310 can perform notification and display thereof in the information terminal apparatus 150 of the user or the information terminal apparatus 400 on the watching side to allow recognition of the user's health condition.

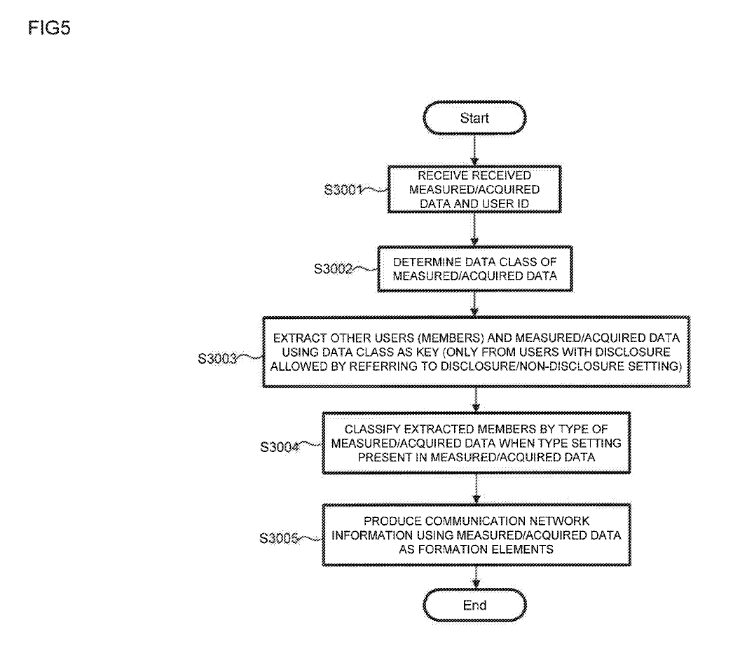

Next, the management apparatus 300 performs communication network production processing based on the received physiological information or the like (S304). FIG. 5 is a diagram showing a detailed flow of the communication network production processing in the present embodiment.

As shown in FIG. 5, when the management apparatus 300 (communication network control section 313) receives the user ID and the physiological information or the like from the information terminal apparatus 150 (S3001), the management apparatus 300 determines the data class of the received physiological information or the like (S3002), refers to the physiological information 321 and the per-user watching information 325 by using the data class as a key, and extracts other users and measured/acquired data (including the living behavior situation and life rhythm) belonging to the data class as network constituent members (S3003).

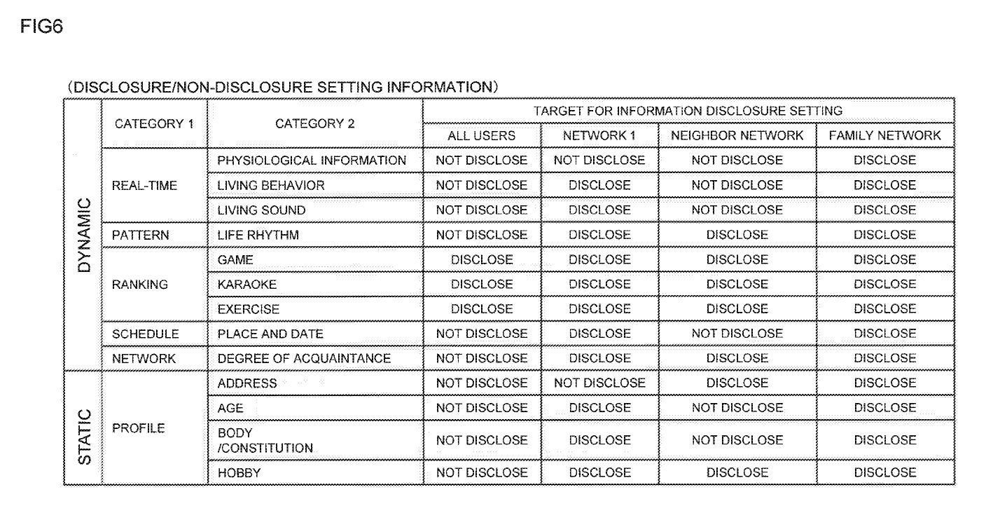

The management apparatus 300 refers to disclosure/non-disclosure setting information of the physiological information, living behavior situation and the like in communication networks shown in FIG. 6 and checks the disclosure/non-disclosure setting of the data class used as the key. The disclosure/non-disclosure setting information stored in the storage unit 320 specifies the disclosure or non-disclosure of information of each category in the network definition information for each user and can be set arbitrarily by the user. In the example of FIG. 6, a network 1 is a communication network group which the user has previously been tied to based on the user's hobby or taste. A neighbor network represents previously grouped other users living in a predetermined neighboring range of the user's address included in the user information. A family network represents previously grouped family members of the user. These communication networks formed by previously tying users based on hobbies or tastes irrespective of the physiological information or the living behavior situation can be previously stored in the user information 327.

The management apparatus 300 uses the data class as the key to extract other users and measured/acquired data (including the living behavior situation and the life rhythm) belonging to the data class as the network constituent members. The extraction is performed only from the users whose disclosure/non-disclosure setting information of the data class used as the key is set to “disclose” (the users with the setting “not-disclose” are excluded).

When group information is set in the data class used as the key, the management apparatus 300 can link each user corresponding to the extracted network constituent member to each group (S3004).

The management apparatus 300 produces communication network information by using the physiological information or the like of the extracted constituent members as network formation elements (S3005). The communication network information can be produced such that the members are divided by their linked groups in the whole network information of the data class.

Returning to FIG. 4, the management apparatus 300 performs processing of providing the produced communication network information to the information terminal apparatus 150 of the user (S305). Specifically, the management apparatus 300 provides a predetermined screen including the communication network information.

When the information terminal apparatus 150 receives the communication network information, the control section 152 performs display control for causing the display section 153 to display a communication network on a screen of predetermined format (S103). When the user selects one of the members (another user) displayed on the communication network screen, the control section 152 activates the communication function such as an e-mail or call to the selected user (S104). The control section 152 performs processing of connecting to the information terminal apparatus 150 of the selected member and controls communication processing through the communication function (S105).

The result of the communication with the other user in the information terminal apparatus is transmitted to the management apparatus 300. The management apparatus 300 uses the received communication result to update the degree of acquaintance representing the degree of closeness between those users (S306).

As described above, the communication network in the present embodiment is dynamically produced by using, as the key, the information changing with each passing hour or each passing day such as the physiological information or the living behavior situation of the user and supports communication between users. The communication network production processing in the present embodiment can include two methods described below.

A first method of production includes dynamically extracting network constituent members according to the physiological information or living behavior situation of the user from the network members tied previously based on their hobbies or tastes irrespective of their physiological information or living behavior situations among all the users participating in the communication management system.

The member group information of the network members tied previously based on hobbies or tastes can be stored as the user information 327 in the storage unit 320, and the user participates in that communication network of his own will. The user can register a communication network which he wants to participate in or can change a communication network through a predetermined screen on the information terminal apparatus 150. The user can also start a communication network and can newly register a communication network which allows other users to participate in and to be tied previously based on hobbies or tastes.

In the first method, to dynamically produce the communication network by using, as the key, the physiological information and living behavior situation of the user changing with each passing hour or each passing day, the user can specify the range of the extraction such that the communication network is formed from members of a single or a plurality of communication networks tied previously based on hobbies or tastes. For example at step S3003 in FIG. 5, the management apparatus 300 can extract members of a single or a plurality of communication networks tied previously based on hobbies or tastes within the specified range by using the data class as the key. The information for specifying the range in a single or a plurality of communication networks consisting of members tied previously based on hobbies or tastes can be stored in the user information 327 for each user.

A second method of production includes dynamically extracting network constituent members according to the physiological information or living behavior situation of the user from all the users participating in the communication management system.

In contrast to the first method, the second method can dynamically extract other users than the users of a single or a plurality of communication networks tied previously based on hobbies or tastes, as network constituent members according to the physiological information or living behavior situation of the user. This can support communication with unacquainted users (users met for the first time).

The first method and the second method can extract, for example, the users whose physiological information or living behavior information were measured or acquired at the same time, as network constituent members. For example, a communication network can be formed of a group of other users who use cleaners within the same time period. In this case, time information attached to the physiological information or the like can be referred to extract, by using the data class as the key, the other users who use cleaners at times considered as the same time or substantially the same time when they fall within a predetermined time range.

The physiological information such as blood pressure may be measured at different measurement times among users. In this case, other users are extracted by using the blood pressure as the key, and network constituent members may be extracted from the users having any past blood pressure measurement result after the previous measurement without specifying any time range. Conversely, a communication network may be formed by excluding the users who do not have the latest blood pressure measurement result in a predetermined time period from the present time.

When the life rhythm is used as the key, at a predetermined time after a life rhythm for one day is produced or at an arbitrary time determined by the action of the user, a communication network relating to the life rhythm can be produced by extracting members from all the users participating in the communication management system or from a single or a plurality of communication networks tied previously based on hobbies or tastes.

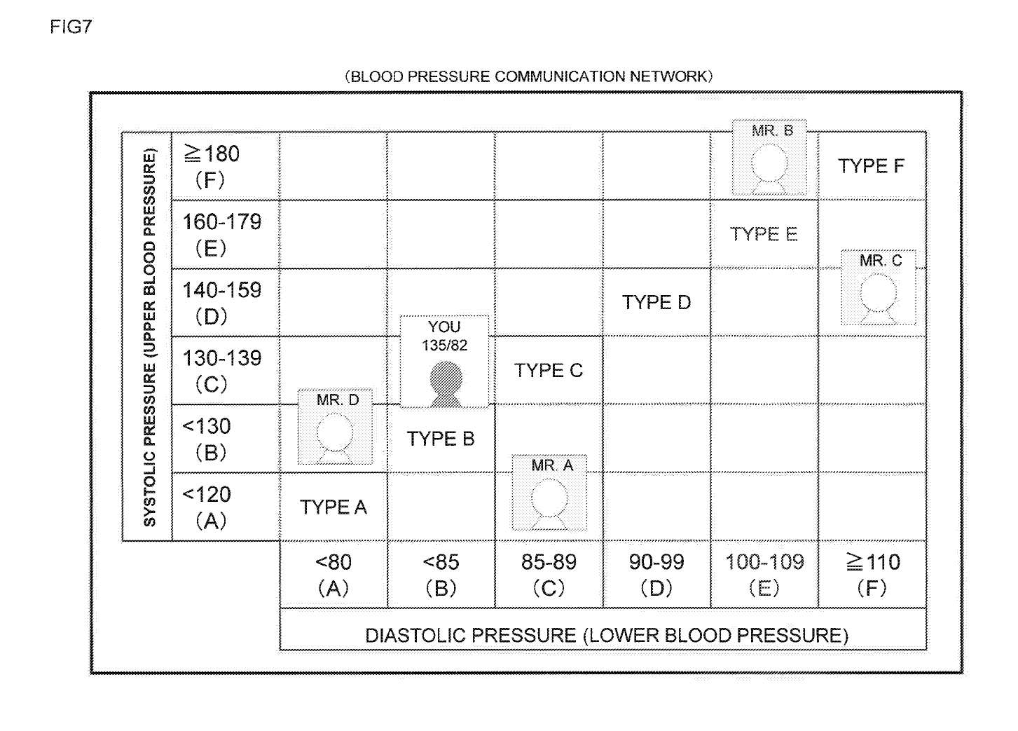

FIG. 7 is a diagram showing an exemplary blood pressure communication network. In the blood pressure communication network shown in FIG. 7, for example, when the blood measurement data of a user (you) is transmitted to the management apparatus 300, the management apparatus 300 uses the blood pressure as the key to extract the other users having blood pressure measurement data as network constituent members and produces the blood pressure communication network. In this case, as described above, the network can be formed by extracting members from all the users participating in the communication management system or from the users in a single or a plurality of communication networks tied previously based on hobbies or tastes. The network can be produced by extracting the network constituent members without specifying any time range or any time of blood pressure measurement or within a specified time range.

In the example of FIG. 7, the vertical axis represents the systolic pressure and the horizontal axis represents the diastolic pressure. On a matrix including display areas (corresponding to blood pressure types A to F) provided by segmenting the systolic pressure and diastolic pressure values by blood pressure type shown in the lower part of FIG. 3, mapping representation is performed such that users of different pressure types are placed. For example, normal high-value blood pressures (type C) are defined as the systolic blood pressure (upper blood pressure) from 130 to 139 and the diastolic blood pressure (lower blood pressure) less than 90, or the systolic blood pressure (upper blood pressure) less than 140 and the systolic blood pressure (lower blood pressure) from 85 to 89 (from Guidelines for the Management of Hypertension 2009 by the Japanese Society of Hypertension). From the viewpoint of protecting privacy, the other users may be searched for and extracted as the network constituent members with reference to the blood pressure type instead of the blood pressure value to produce the blood pressure communication network.

Each user in the blood pressure communication network can be displayed by an icon including the name and the like and the icon can be selected in a touch-panel manner. Once an arbitrary user is selected, an e-mail can be sent or a call can be made to the selected user over the videophone or the like. The icon of the user of interest is displayed as “you.” The icons representing the users including the user (you) are placed at the associated display areas segmented by blood pressure type.

Although the example of FIG. 7 shows the blood pressure communication network including the users of all the blood pressure types, a blood pressure communication network for each blood pressure type can be provided, for example.

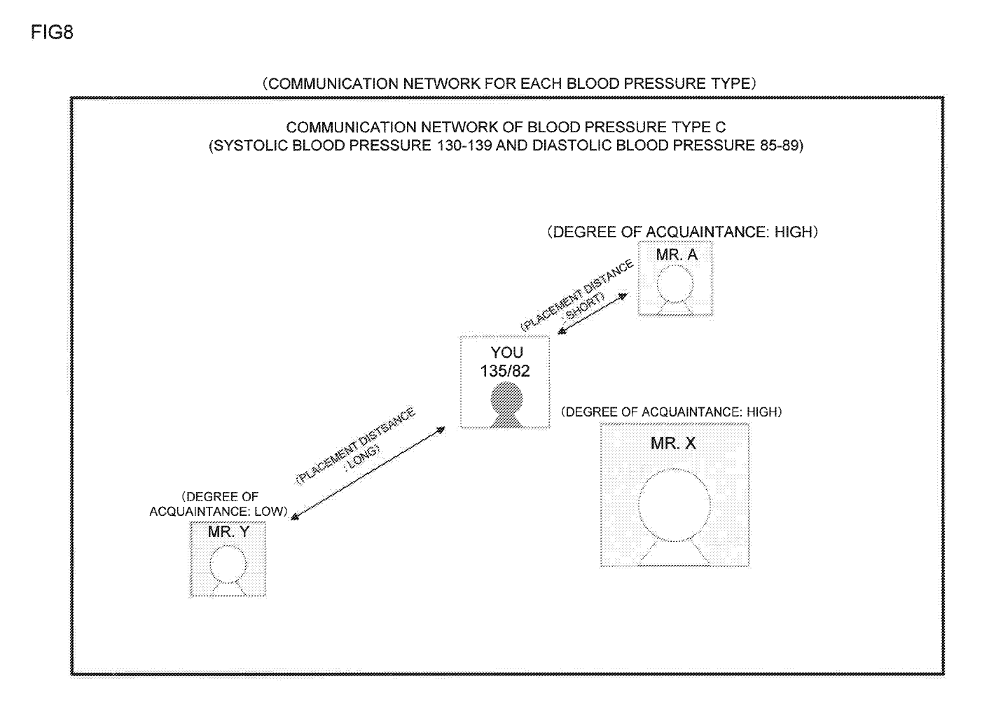

FIG. 8 shows an example of the blood pressure communication network for each blood pressure type. As shown in FIG. 8, for example, only the other users having blood pressure measurement data belonging to the blood pressure type C are displayed as network constituent members. As the degree of acquaintance is lower between the user and another user, the other user can be placed at a longer distance between the icon of the user and the icon of the other user. As the degree of acquaintance is higher, the other user can be placed at a shorter distance between the icons. Alternatively, the size of an icon can be used to represent the degree of acquaintance between the user and another user instead of the distance between icons. As the degree of acquaintance with another user is higher, the other user can be displayed by a larger icon. As the degree of acquaintance with another user is lower, the other user can be displayed by a larger icon.

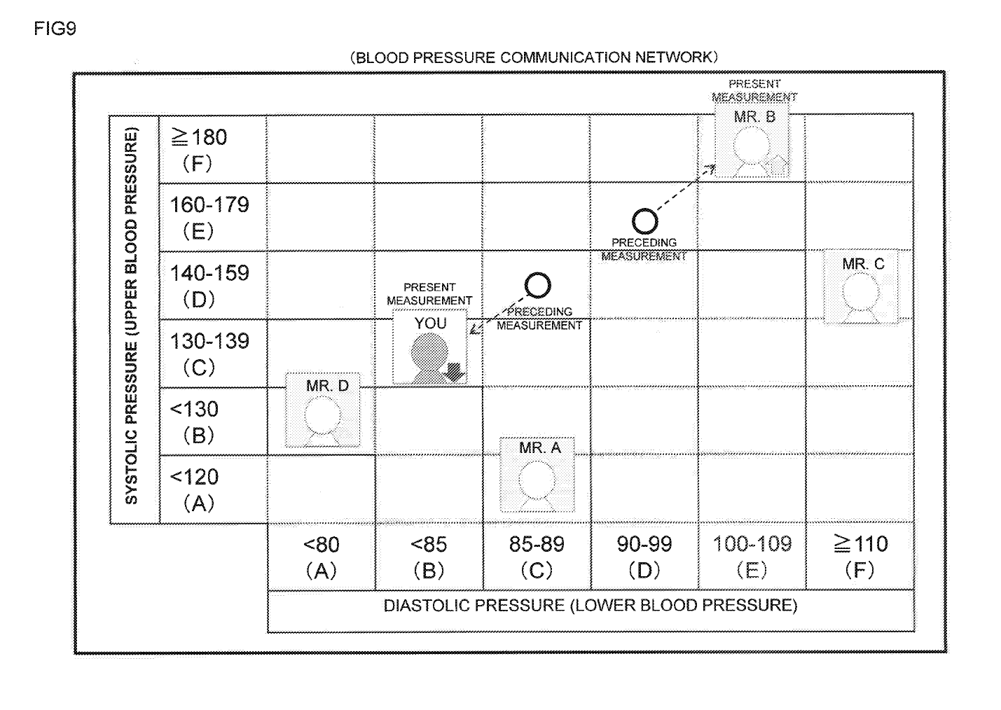

FIG. 9 is a diagram showing a blood pressure communication network provided by adding changes in blood pressure measurement data between the past and present to the blood pressure communication network shown in FIG. 7.

In FIG. 9, the user (you) belonged to the blood pressure type D in the preceding measurement but changes to the blood pressure type C in the present measurement result. In contrast, Mr. B belonged to the blood pressure type E in the preceding measurement but changes to the blood pressure type F in the present measurement. Such changes in blood pressure type, that is, health condition estimated from the blood pressure are displayed in the blood pressure communication network to allow the user to have a conversation with a user different from the other users whom the user communicated with in the previous blood pressure type or to have communication with a user whose blood pressure type was degraded, thereby supporting more active communication.

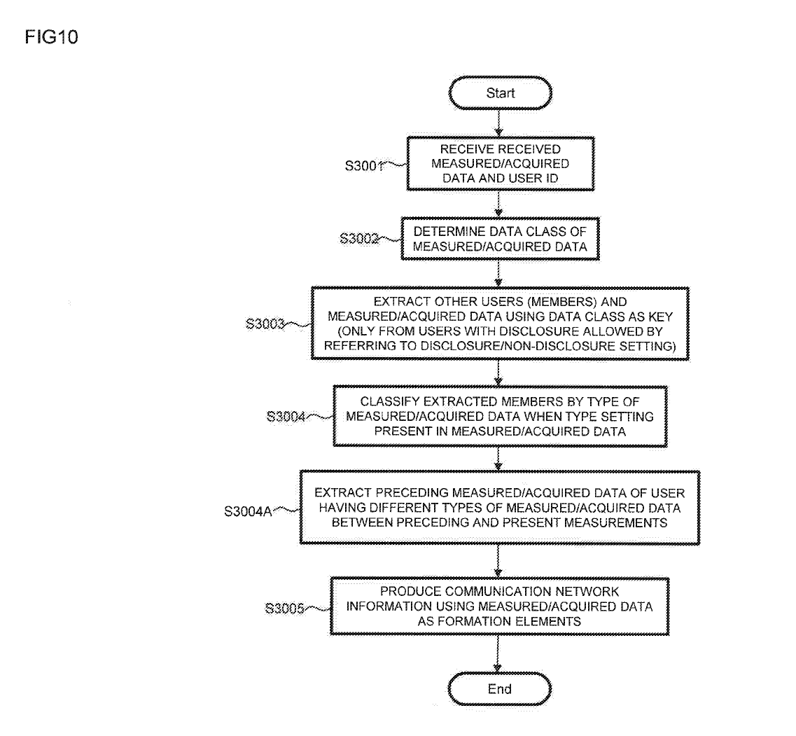

FIG. 10 is a diagram showing a flow of processing of producing a communication network including blood pressure changes. FIG. 10 includes step S3004A added to the production processing flow shown in FIG. 5, and the other steps are identical to those in FIG. 5.

At step S3004A, the management apparatus 300 (communication network control section 313) refers to the present blood pressure type and the preceding blood pressure type of one user of the network constituent members extracted by using the data class “blood pressure” as the key and determines whether or not the preceding and present blood pressure types are different. If they are different, the management apparatus 300 extracts both the latest (present) blood pressure type and the past (preceding) blood pressure type of the one user from the per-user watching information 325.

Then, at step S305, the management apparatus 300 places the icon associated with the present blood pressure type as the icon of the user at the display area corresponding to that blood pressure type, and displays change status information such as a symbol or an arrow indicating the change from the preceding blood pressure type to the present blood pressure type across the display areas of the blood pressure types. Similar display is performed for the blood pressure change of each user.

As described above, the present embodiment includes not only displaying the blood pressure measurement data dynamically acquired for each user on the communication network, but also extracting the first blood pressure measurement data acquired in the preceding measurement and the second blood pressure measurement data acquired in the present measurement for each user to produce the blood pressure change communication network information including the status change from the first blood pressure measurement data to the second blood pressure measurement data. This can allow visual recognition of changes in the user's health condition and changes in health condition of other users, and the communication can be promoted by the health condition changes.

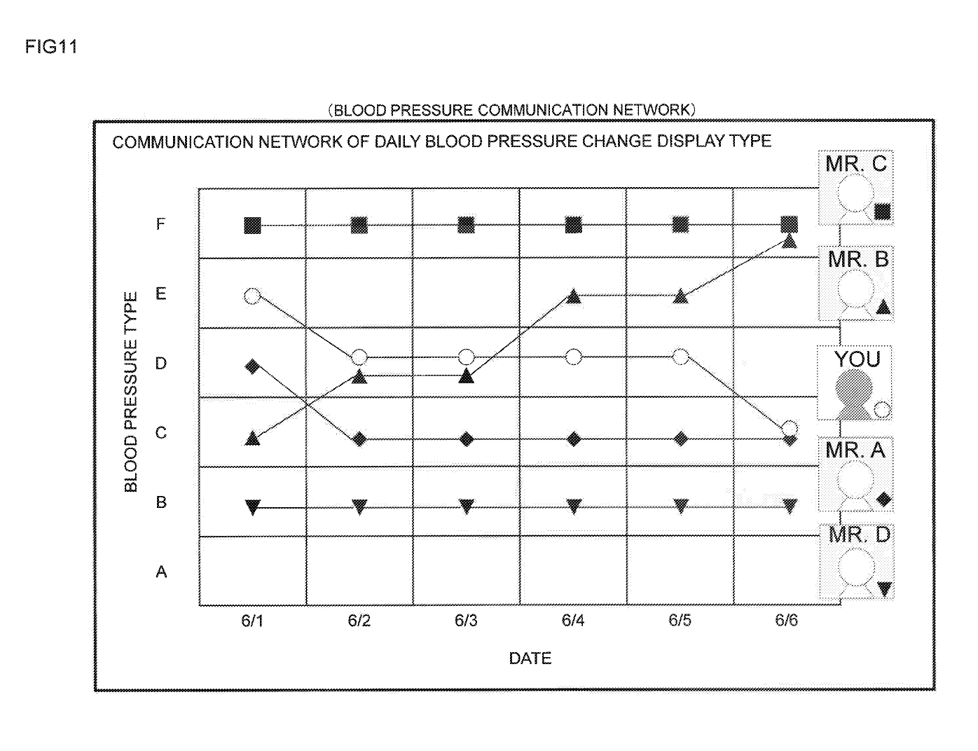

FIG. 11 is a diagram showing an exemplary communication network of daily blood pressure change display type. In the example of FIG. 11, the vertical axis represents the blood pressure type and the horizontal axis represents date, and daily blood pressure changes of each user are shown in graphical form. Changes in blood pressure type of each user are indicated as a line graph, and the line graph is presented in association with the icon of each user. Similarly to the example of FIG. 7, once the icon representing each user is selected, communication such as an e-mail or a call to the selected user can be performed through the information terminal apparatus 150. The communication network including such blood pressures (physiological information) dynamically acquired in daily life and arranged in temporal sequence is produced to enable visual recognition of changes in health condition of each user back to the past, thereby further promoting communication.

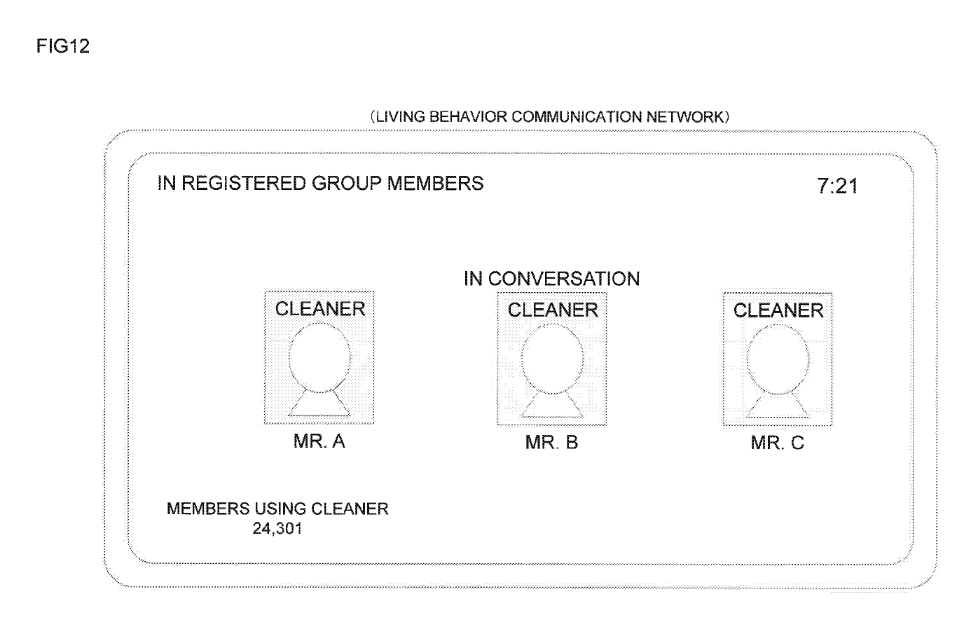

FIG. 12 is a diagram showing an exemplary living behavior communication network of the present embodiment. While the user (you) is using a cleaner, the management apparatus 300 can know the user's living behavior situation of “using cleaner.” As shown in the processing flows of FIG. 4 and FIG. 5, the data class “using cleaner” is used as the key to produce a communication network of which network constituent members are the other users who are using cleaners at the same time.

In the example of FIG. 12, the number of persons who are using cleaners of all the users participating in the communication management system and the present time are displayed. The network constituent members of the living behavior communication network are the other users extracted from members in a single or a plurality of networks tied previously based on hobbies or tastes of the user (you). Of them, the user (you) and Mr. B are making a call over the videophone.

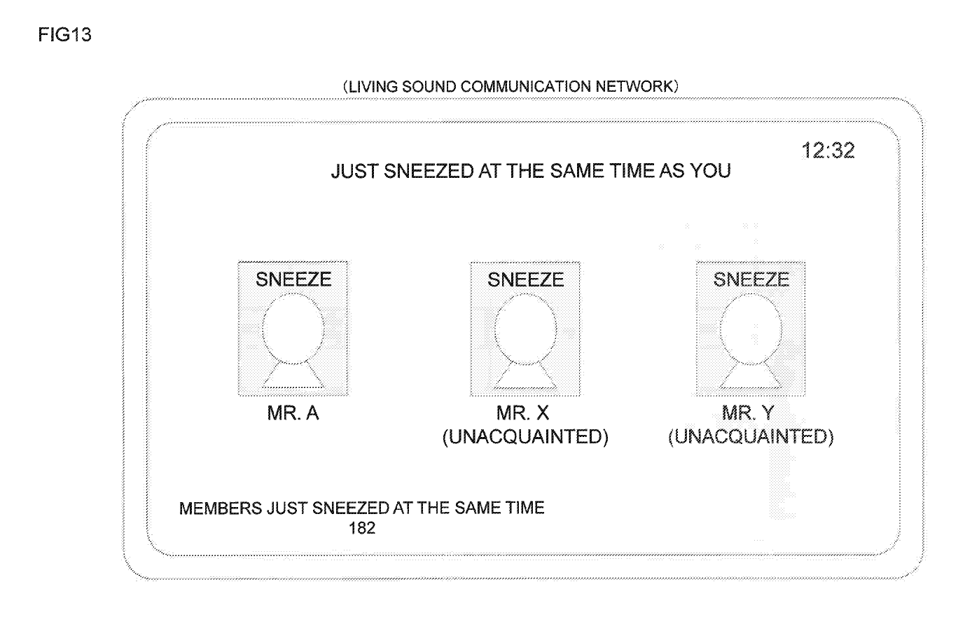

FIG. 13 shows an exemplary living sound communication network. In the example of FIG. 13, when the user (you) sneezes, the management apparatus 300 can know the user's living behavior situation of “sneezing.” As shown in the processing flows of FIG. 4 and FIG. 5, the data class “sneeze” is used as the key to produce a communication network of which network constituent members are the other users who sneezed at the same time.

In the example of FIG. 13, similarly to the living behavior communication network of FIG. 12, the number of persons who sneezed of all the users participating in the communication management system and the present time are displayed. The network constituent members of the living sound communication network are the other users extracted from members in a single or a plurality of networks tied previously based on hobbies or tastes of the user (you).

FIG. 14 shows an exemplary life rhythm communication network. As described above, the life rhythm information includes living behavior situations of each user for one day represented in predetermined units of time and arranged in temporal sequence.

The management apparatus 300 can use the life rhythm information as the network formation element to produce, for example, life rhythm communication network information showing the life rhythm information of the same day of the user (you) and a plurality of other users of a single or a plurality of networks tied previously based on hobbies or tastes. In the example of FIG. 14, the icon of each user in the life rhythm communication network can be presented in non-active display (gray) according to time setting, setting of telephone not-allowed through operation input by the user, or living behavior situation such as during sleep.

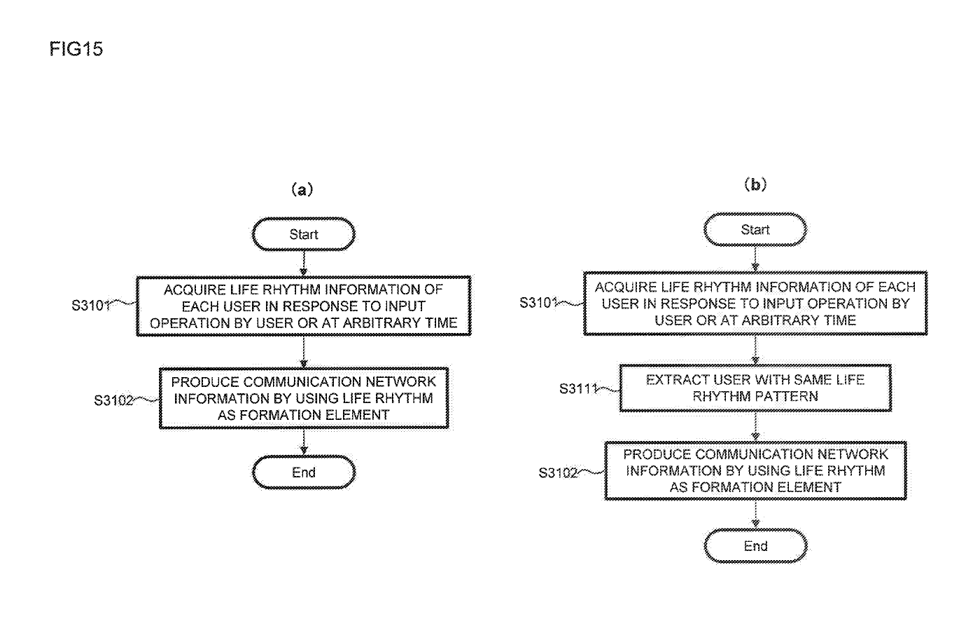

FIG. 15 is a diagram showing flows of life rhythm communication network production processing. Each processing shown in FIG. 15 (a) and FIG. 15(b) represents a detailed flow of step S304 shown in FIG. 4.

In FIG. 15 (a), in response to input operation by the user through the information terminal apparatus 150 (for example, operation of a button for selecting life rhythm communication network production), the management apparatus 300 can extract life rhythm information including living behavior situations of each user up to the present time from all the users participating in the communication management system or the users of a single or a plurality of communication networks tied previously based on hobbies or tastes of the user (you) to produce a life rhythm communication network showing the life rhythm information for each user.

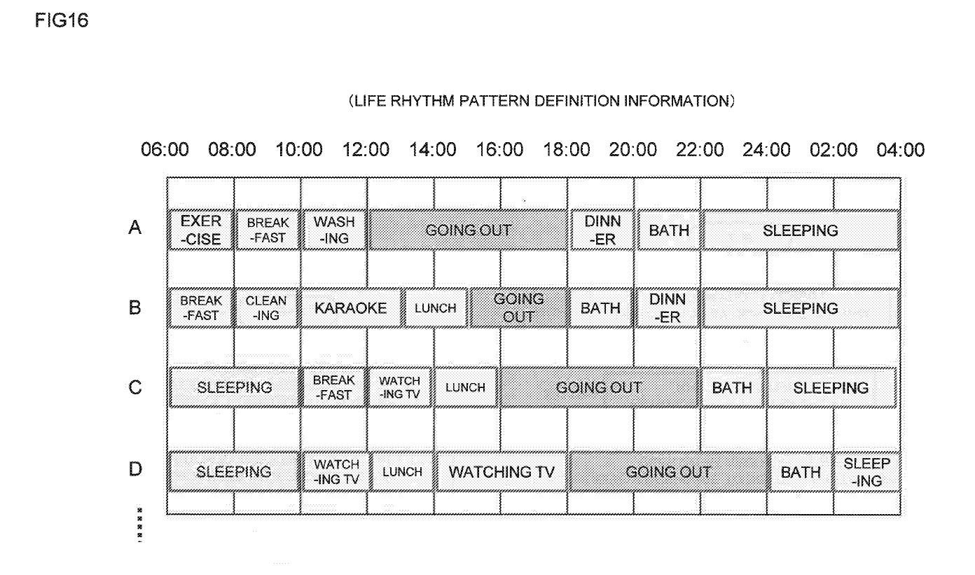

FIG. 16 shows life rhythm pattern definition information. For example, a healthy life rhythm, an unhealthy life rhythm and the like can be previously defined in stages as patterns (Patterns A, B, . . . ) Based on the pattern definition information for classifying life rhythm information into the predetermined patterns, the management apparatus 300 can determine the life rhythm pattern of the life rhythm information for each user.

In FIG. 15(b), it is determined which life rhythm pattern the user belongs to, and for example, the other users who match the determined pattern can be extracted as network constituent members to produce a life rhythm communication network.

FIG. 17 is a diagram showing life rhythm changes and pattern changes, and illustrates an example in which the daily life rhythms of the user are arranged in date order along the vertical axis and each life rhythm is assigned a life rhythm pattern as status change information based on the pattern definition information.

The life rhythm change information including the determined life rhythm patterns arranged for the respective days is produced. This allows the user (you) to know that the user (you) are living a healthy life or going to live a healthy life from the dynamic communication network produced according to the physiological information or living behavior situation through the communication management system. When the life pattern is degraded, the user (you) can know that the user (you) is living an unhealthy life or going to fall into an unhealthy life pattern.

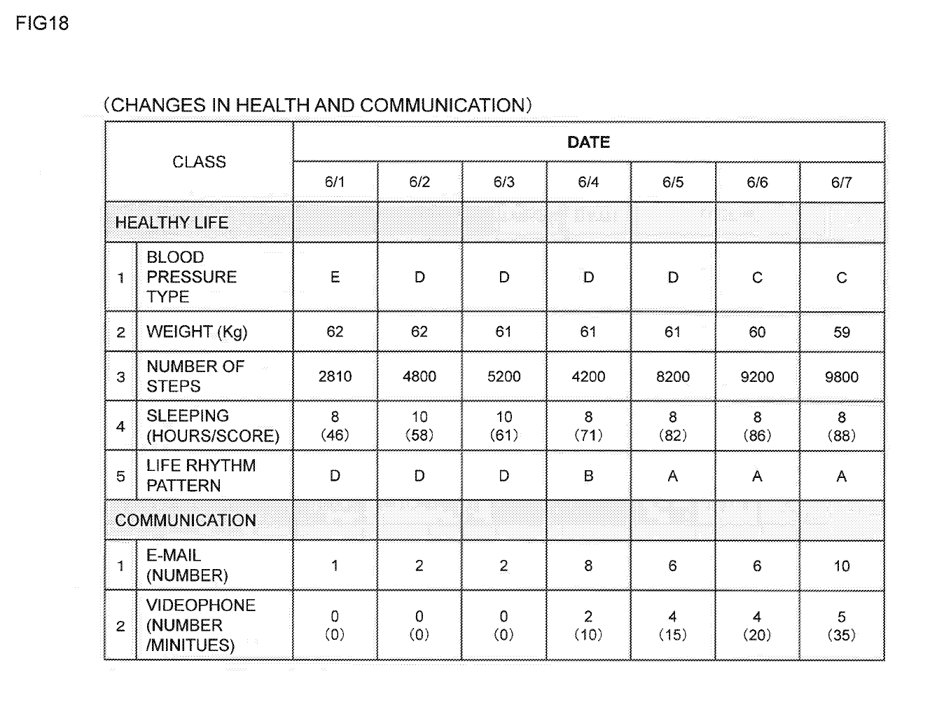

FIG. 18 is a diagram showing exemplary changes in health and communication. The example of FIG. 18 presents information including the blood pressure type of the physiological information, weight, number of steps, sleeping time, and life rhythm of one user for each day together with the degree of communication for each day (the number of e-mails, the number and duration of calls over the videophone).

Since the changes in daily physiological information and living behavior and the changes in degree of communication can be obtained as in the example of FIG. 18, a healthier condition resulting from an increased amount of exercise and reduced blood pressure or weight can be found from the life rhythm changing toward a better pattern, for example. It can also be seen that, as the degree of communication is higher, the life rhythm changes toward a better pattern or a healthier condition is achieved. In other words, it can be seen that more active communication with the other users through the communication network of the present embodiment leads to a healthier life of the user, which can further promote the communication.

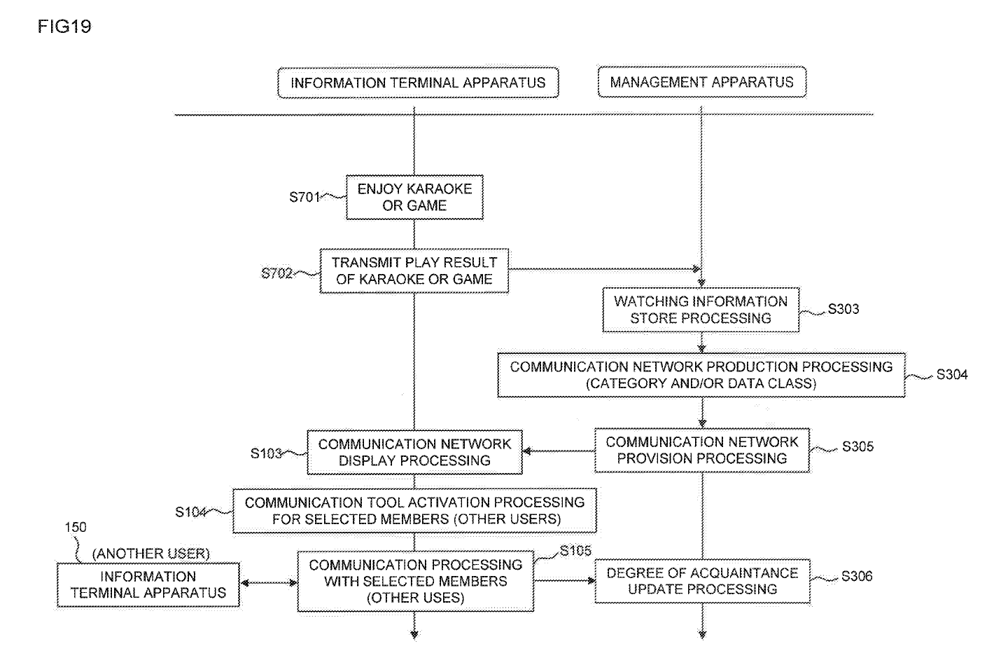

FIG. 19 is a diagram showing a flow of communication network production processing (based on ranking) according to Modification 1 of the present embodiment.

As shown in FIG. 3, a communication network can be produced by using each data class of the ranking in the network definition information as the network formation element. The user can activate the karaoke function or game function installed as applications on the information terminal apparatus 150 from a predetermined screen to enjoy karaoke or games (S701).

The result of the user enjoying the karaoke function or game function, that is, the play result such as the score of karaoke or games or the time spent on the play can be transmitted from the information terminal apparatus 150 to the management apparatus 300 (S702). The management apparatus 300 can store the received play result in the storage unit 320 for each data class (S303) and produce communication network information by using the second category or each data class of the network definition information 326 as the key (S304). Since the other steps in FIG. 19 are identical to the steps described in FIG. 4, the same reference numerals are given and description thereof is omitted.

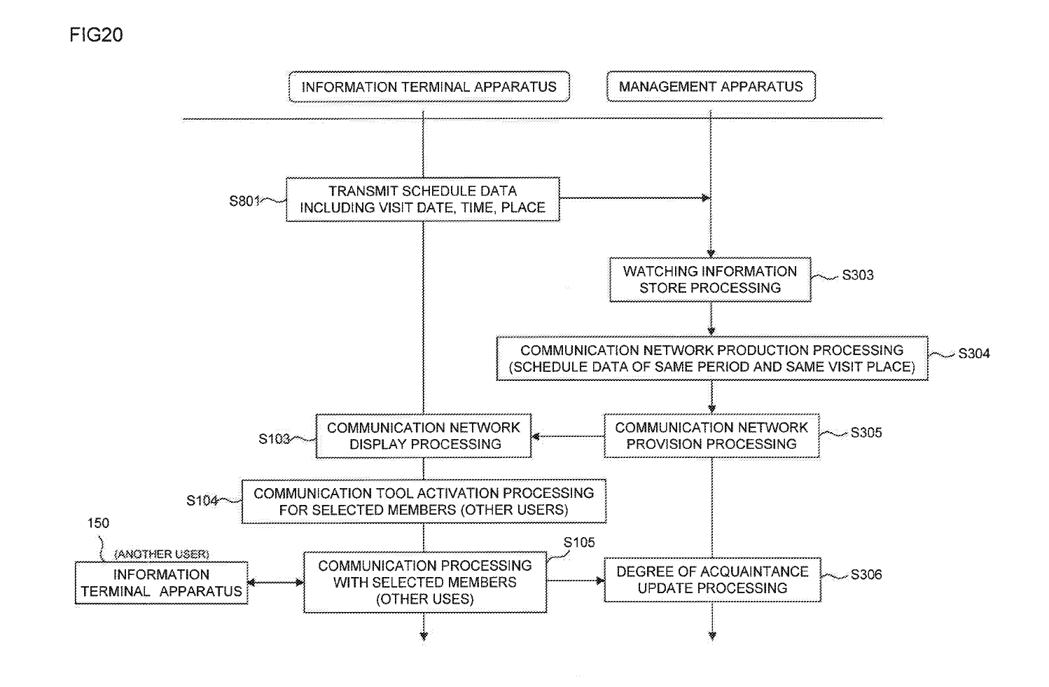

FIG. 20 is a diagram showing a flow of communication network production processing (based on schedule) according to Modification 2 of the present embodiment.

As shown in FIG. 3, a communication network can be produced by using each data class (visit date, time, and place) of the schedule in the network definition information as the network formation element. The user can input the visit date and time, and visit place to a schedule screen on the information terminal apparatus 150. The information terminal apparatus 150 transmits the input visit date and time, and visit place as schedule information to the management apparatus 300 (S801).

Upon reception of the schedule information of the user, the management apparatus 300 can store that information in the storage unit 302 for each data class (S303) and use each data class of the network definition information 326 as the key to produce communication network information (S304). For example, the management apparatus 300 can produce a communication network by using the visit place as the network formation element or can produce a communication network by using the visit date, time, and place as the network formation elements to extract the other users having schedule data matching such network formation elements within a predetermined time range (for example, within few days) as constituent members. Since the other steps in FIG. 20 are identical to the steps described in FIG. 4, the same reference numerals are given and description thereof is omitted.

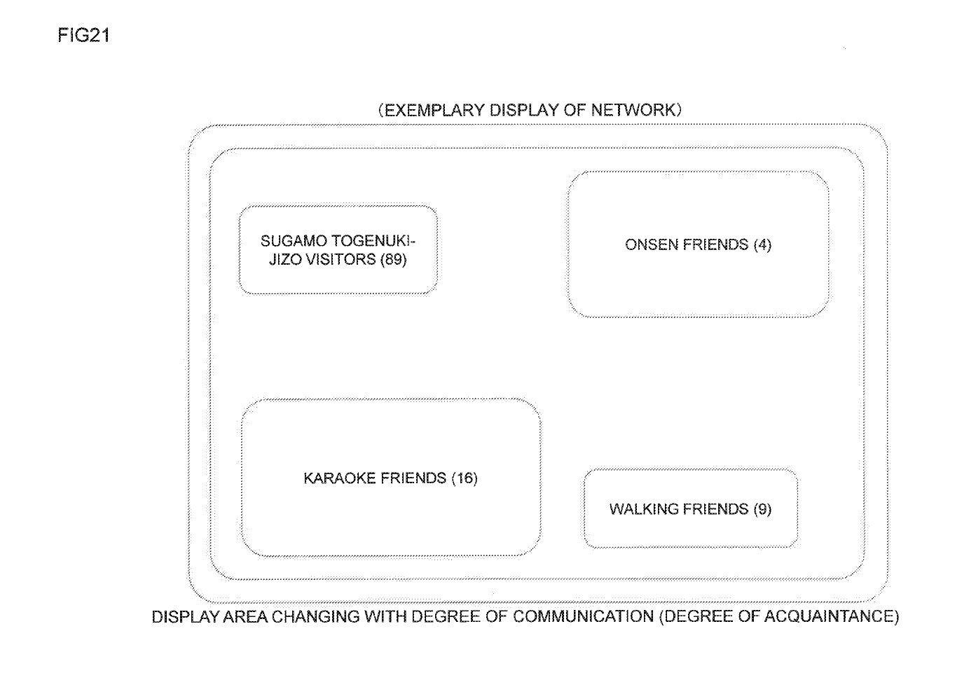

FIG. 21 is a diagram showing an example of a single or a plurality of communication network groups which the user has previously been tied to based on hobbies or tastes described above. The example of FIG. 21 displays communication network groups participated in based on the user profile registered by the user or registered by the user of his own will. The display is controlled such that the communication network group with a higher degree of acquaintance with each member of the network group has an area larger than that of the other communication network group with a lower degree of acquaintance.

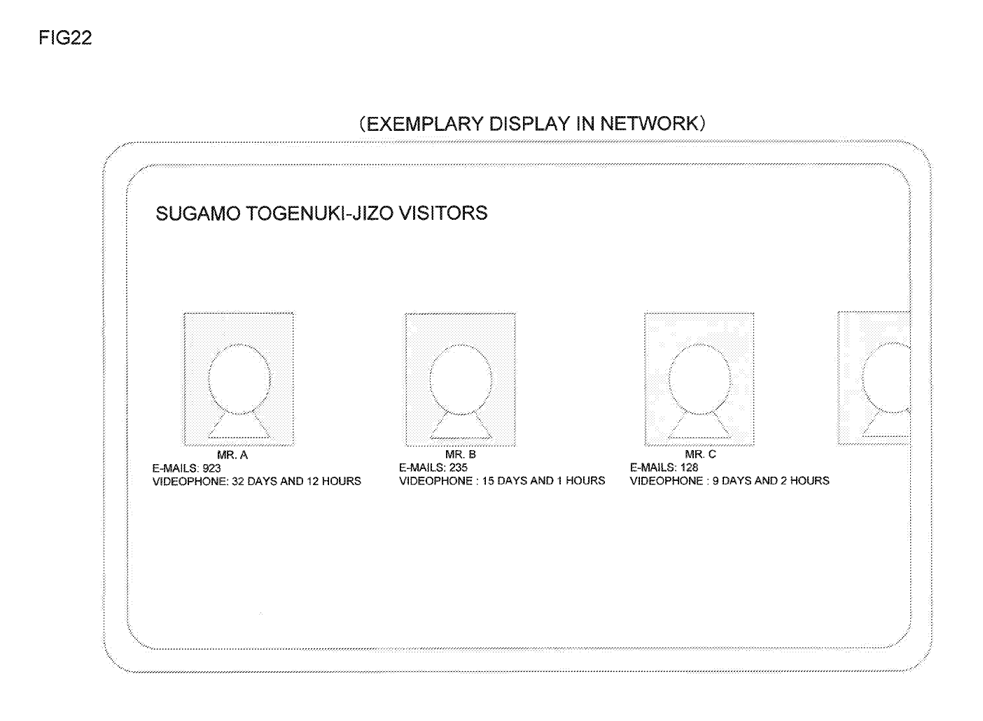

The user can select each communication network group shown in FIG. 21 on the information terminal apparatus 150 to display the icons of members of the selected communication network group as shown in FIG. 22. The information terminal apparatus 150 can display the name of each member and the degree of communication (such as the number of c-mails and duration of calls) near the icon of the member as the degree of acquaintance. The information about the degree of acquaintance of other members can be acquired from the management apparatus 300 when that communication network group is selected, or the degree of acquaintance for each member stored previously in the information terminal apparatus 150 can be used.

The communication management apparatus of the present embodiment is connected to the plurality of information terminal apparatuses 150 and provides the communication network information for communication of the users between the plurality of information terminal apparatuses 150. The communication management system stores for each of the users the physiological information and/or the living behavior information of the user, acquired dynamically in daily life, and produces the communication network information being associated with other users to one of the uses based on the physiological information and/or living behavior information of one of the users. The communication network information is produced by using each of the dynamically acquired physiological information and/or the living behavior information of that user as the network formation element. Such a configuration allows active communication between the users to be promoted by the health condition or living behavior, thereby providing an environment in which the communication between the users relating to the health or life can lead to improved health and mutual watching of the users.

In the above description, the user information 327, the group information of data class shown in the lower part of FIG. 3 and the like can be stored on the information terminal apparatus 150. In this case, for example, the group information of data class corresponding to the blood pressure measurement data received from the physiological information measurement apparatus 100 can be determined, and that group information (blood pressure type) can be transmitted to the measurement apparatus 300 together with the blood pressure measurement data. The user information 327 can include, for example, contact information such as the e-mail addresses and telephone numbers of other users, so that the user information 327 is also stored on the information terminal apparatus 150 to realize smooth communication with the constituent members of the communication network dynamically produced according to the physiological information and/or living behavior situation.

Each of the functions of the communication management system (management apparatus 300) of the present embodiment can be configured as a program. For example, a program for each function of the communication management system can be stored on an auxiliary storage apparatus, not shown, of a computer, a control unit such as a CPU can read the program stored on the auxiliary storage apparatus to a main storage apparatus, and the program read to the main storage apparatus can be executed by the control unit to cause the computer to perform the function of each component in the present invention. Thus, the computer on which the program for each function of the communication management system of the present embodiment is installed can operate as a computer apparatus which achieves each function of the communication management system of the present embodiment.

The program may be recorded on a computer readable recording medium and provided for a computer. Examples of the computer readable recording medium include optical disks such as a CD-ROM, phase-change optical disks such as a DVD-ROM, magneto-optical disks such as a Magnet-Optical (MO) disk and Mini Disk (MD), magnetic disks such as a floppy Disk® and removable hard disk, and memory cards such as a compact Flash®, smart media, SD memory card, and memory stick. Hardware apparatuses such as an integrated circuit (such as an IC chip) designed and configured specifically for the purpose of the present invention are included in the recording medium.

Although the preferred embodiment of the present invention has been described, that embodiment is illustrative and is not intended to limit the scope of the present invention. The novel embodiment can be implemented in various other forms, and various omissions, substitutions, and modifications can be made thereto without departing from the spirit or scope of the present invention. The embodiment and its variations are encompassed within the spirit or scope of the present invention and within the invention set forth in the claims and the equivalents thereof.
100 PHYSIOLOGICAL INFORMATION MEASUREMENT APPARATUS
101 COMMUNICATION SECTION
102 SENSOR MODULE
150 INFORMATION TERMINAL APPARATUS
200 LIVING BEHAVIOR ACQUIREMENT APPARATUS
201 SENSOR APPARATUS
202 PROCESSING SECTION
203 COMMUNICATION SECTION
300 MANAGEMENT APPARATUS (COMMUNICATION MANAGEMENT APPARATUS)
310 CONTROL APPARATUS
311 BEHAVIOR ESTIMATION SECTION
312 HEALTH CONDITION DETERMINATION SECTION
313 COMMUNICATION NETWORK CONTROL SECTION
320 STORAGE UNIT
321 PHYSIOLOGICAL INFORMATION
322 BEHAVIOR INFORMATION
323 BEHAVIOR PATTERN KNOWLEDGE INFORMATION
324 HEALTH DETERMINATION KNOWLEDGE INFORMATION
325 PER-USER WATCHING INFORMATION
326 NETWORK DEFINITION INFORMATION
327 USER INFORMATION
400 INFORMATION TERMINAL APPARATUS

1. A communication management system connected to a plurality of information terminal apparatuses and configured to provide communication network information for communication of users between the plurality of information terminal apparatuses, comprising:
a storage apparatus configured to store for each of the users physiological information and/or living behavior information of the user, acquired dynamically in daily life; and
a control apparatus configured to produce the communication network information being associated with other users to a first user based on the physiological information and/or living behavior information of the first user,
wherein the control apparatus is configured to produce the communication network information by using each of the dynamically acquired physiological information and/or living behavior information of the first user as a network formation element.

2. The communication management system according to claim 1, wherein the control apparatus is configured to extract, from the physiological information acquired dynamically in each of users corresponding to network constituent members of the communication network information, first physiological information acquired in preceding measurement and second physiological information acquired in present measurement for each of the users, and to produce the communication network information including a status change representing a change from the first physiological information to the second physiological information.

3. The communication management system according to claim 1 or 2, wherein the control apparatus is configured to produce, based on definition information for grouping the physiological information into a plurality of types, the communication network information including an display area of each of the types, each user being mapped to an associated one of the display areas according to the type.

4. The communication management system according to any one of claims 1 to 3, wherein the control apparatus is configured to use the living behavior information acquired dynamically in each of users corresponding to network constituent members of the communication network information to produce daily life rhythm information including the living behavior information arranged in temporal sequence for each of the users, and
the control apparatus is configured to produce, by using the produced life rhythm information as a network formation element, the communication network information in which the life rhythm information of each of the plurality of users is displayed.

5. The communication management system according to claim 4, wherein the control apparatus is configured to determine a life rhythm pattern of the life rhythm information of the first user based on definition information for grouping the life rhythm information into predetermined patterns, and
the control apparatus is configured to produce life rhythm change information in which the life rhythm information and the life rhythm pattern of the first user in each of a plurality of days is arranged.

6. The communication management system according to any one of claims 1 to 5, wherein the information terminal apparatus includes:
display means for displaying a predetermined network screen based on the communication network information produced by the control apparatus; and
communication means for communicating with another user belonging to a communication network of the communication network information displayed on the communication network screen.

7. A program executed by a computer connected to a plurality of information terminal apparatuses, the computer being configured to provide communication network information for communication of users between the plurality of information terminal apparatuses, the program comprising:
a first function of storing for each of the users physiological information and/or living behavior information of the user, acquired dynamically in daily life; and
a second function of producing the communication network information being associated with other users to a first user based on the physiological information and/or living behavior information of the first user,
wherein the second function is configured to produce the communication network information by using each of the dynamically acquired physiological information and/or living behavior information of the first user as a network formation element.
